(19) United States
(12) Patent Application Publication
(10) Pub. No.: US 2021/0069500 A1
(43) Pub. Date: Mar. 11, 2021

(54) SYSTEMS, DEVICES AND METHODS FOR PLACEMENT AND FIXATION OF NEUROMODULATION SYSTEM IN COMBINATION WITH A DECOMPRESSION PROCEDURE

(71) Applicant: SynerFuse, Inc., Minneapolis, MN (US)

(72) Inventors: Gregory F. Molnar, Blaine, MN (US); Nazmi Peyman, Glen Allen, VA (US); Beth A. Lindborg, St. Paul, MN (US); Kathleen W. Hill, St. Paul, MN (US); Kyle R. Grube, Minneapolis, MN (US); Michael C. Park, Excelsior, MN (US); Matthew A. Hunt, Minneapolis, MN (US); Justin D. Zenanko, Minnetrista, MN (US); Susan Alpert, Washington, DC (US)

(21) Appl. No.: 17/101,800

(22) Filed: Nov. 23, 2020

Related U.S. Application Data
(62) Division of application No. 16/743,786, filed on Jan. 15, 2020.
(60) Provisional application No. 62/793,319, filed on Jan. 16, 2019.

(51) Int. Cl. A61N 1/32; A61N 1/36; A61N 1/04; A61N 1/05

(52) U.S. Cl. CPC A61N 1/326; A61N 1/0551; A61N 1/048; A61N 1/3605

(57) ABSTRACT

The present invention provides various embodiments of neuromodulation systems, and improvements thereof, capable of being implanted at a spinal treatment site and capable of being implanted at the same time and/or in combination with a spinal procedure being performed at the spinal treatment site. The present invention further includes improvements in the number and types of neuromodulation therapies that can be implanted at the spinal treatment site and improvements to the neuromodulation systems used for delivering such neuromodulation therapies.

### CROSS-REFERENCE TO RELATED APPLICATIONS

This application is a divisional of U.S. Utility application Ser. No. 16/743786, filed Jan. 15, 2020 and titled IMPROVED NEUROMODULATION THERAPIES AND NEUROMODULATION SYSTEMS, and also claims the benefit of U.S. Provisional Patent Application No. 62/793,319, filed Jan. 16, 2019 and titled NEW NEUROMODULATION THERAPIES AND IMPROVED NEUROMODULATION SYSTEMS, the entirety of which are hereby incorporated by reference.

### STATEMENT REGARDING FEDERALLY SPONSORED RESEARCH OR DEVELOPMENT

Not Applicable

### BACKGROUND OF THE INVENTION

Neuromodulation for the treatment of chronic spinal pain is a procedure that has been in use for decades. The procedure is generally prescribed to a patient only after they have gone through a spinal procedure to correct the supposed source of the pain and, after weeks, months and perhaps years of continued chronic pain and pain therapy through medications, including opioids, the patient may finally be prescribed neuromodulation for the treatment of chronic pain after failed back surgery.

Without being bound by theory, the present invention is based upon the premise that many patients who suffer from chronic back pain, such as those who suffer for a long enough period of time or due to the severity of their particular condition, are also separately suffering from neuropathic pain that cannot be corrected by spinal surgery.

In such a case it is a misnomer to say that a patient is suffering from “failed back surgery” but more accurately that the back surgery simply does not address the neuropathic pain that may have been in place prior to the back surgery.

The present invention provides a method for combining the implantation of a spinal treatment device with the implantation of a neuromodulation device, or at least a neuromodulation lead of a neuromodulation device, into a single combination procedure performed at the spinal treatment site. The present invention provides the potential to treat both back stabilization issues and neuropathic pain issues and other types of pain and anatomical treatment and recovery issues in a single procedure, reduce post-operative hospital stay times, perhaps having the additional benefit of minimizing the amount of pain medications, including opioids and other pain medications, that a patient requires in order to manage chronic back pain, resulting in quality of life improvements for the patient. Perhaps also resulting in a reduction in the time of healing and improved quality of life relative to existing therapies.

### SUMMARY OF THE INVENTION

A neuromodulation procedure in accordance with the present invention is performed at a spinal treatment site. The neuromodulation procedure includes the placement of one or more neurostimulation leads at one or more target spinal levels, and more specifically, at one or more nerve targets or other anatomical targets at or near the spinal treatment site.

The neurostimulation leads include a distal portion having one or more electrodes positioned at the distal portion, the neurostimulation leads further include a proximal portion capable of electrically coupling to an implantable pulse generator. The neurostimulation lead further includes one or more electrically conductive wires capable of receiving an electrical signal in a distal portion, when electrically coupled to a pulse generator. The neurostimulation leads, when coupled to an implantable pulse generator, are then capable of delivering an electrical signal via the electrodes to a nerve target or other anatomical target.

The procedure for placing of the neurostimulation leads may include placing the distal segment of one or more neurostimulation leads at the corresponding one or more nerve targets or other anatomical targets such that one or more electrodes of the neurostimulation lead is in therapeutic proximity to the target. When the neuro stimulation lead is coupled to an implantable pulse generator and an electrical signal is delivered to the target via the electrodes of the lead or leads that are electrically coupled to the implantable pulse generator results is neuromodulation of the nerve or other anatomical target.

The neuromodulation procedure may further include routing of the proximal portion of the neurostimulation lead to the implantable pulse generator. The implantable pulse generator may be placed during the spinal procedure in an anatomical location that is dependent upon the particular treatment procedure performed or dependent upon physician preference or dependent upon patient preference or some combination thereof.

Once the implantable pulse generator has been electrically coupled to the leads, the pulse generator can be activated to deliver, via the one or more neurostimulation leads, a neuromodulation therapy to a target at or near the spinal treatment site various embodiments and improvements to which are provided below.

The description of the invention and is as set forth herein is illustrative and is not intended to limit the scope of the invention. Features of various embodiments may be combined with other embodiments within the contemplation of this invention. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

### BRIEF DESCRIPTION OF THE DRAWINGS

FIG. 1A illustrates one embodiment of a neuromodulation system for the treatment of axial pain by innervation of the facet joint.

FIG. 1B is a section of FIG. 1A.

FIG. 2A illustrates one embodiment of a neuromodulation system for the treatment of axial pain by innervation of the facet joint.

FIG. 2B is a section of FIG. 2A.

FIG. 3 illustrates one embodiment of a neuromodulation system for the treatment of axial pain by innervation of the facet joint.

FIG. 4 illustrates one embodiment of an implantable neuromodulation therapy system for the neuromodulation of the nerves inside the spinal disc to treat discogenic pain.

FIG. 5 illustrates one embodiment of an implantable neuromodulation therapy system for the neuromodulation of muscle nerves.

FIG. 6 illustrates one embodiment of an implantable neuromodulation therapy system enabling lead placement adjacent to lateral and anteriolateral parts of the spinal cord in order to modulate spinal tracts.

FIG. 7 illustrates one embodiment of a neuromodulation system for targeting the interbody space for the stimulation of bone growth.

FIG. 8 illustrates an alternative embodiment of one or more leads placed along a lead pathway for targeting the interbody space for the stimulation of bone growth.

FIG. 9 illustrates an embodiment of an implantable neuromodulation system implanted using a minimally invasive procedure during a spinal decompression procedure.

FIG. 10 illustrates one embodiment of a mesh lead design wherein the distal portion of the lead has a mesh shape to provide a broader area of neuromodulation energy to the interbody space.

FIG. 11 illustrates an embodiment of an acute neuromodulation system for any of the various neuromodulation therapies described above.

FIG. 12A illustrates one embodiment of an improved coupling element for electrically coupling a proximal portion of a lead to an implantable pulse generator.

FIG. 12B illustrates one embodiment of an improved coupling element for electrically coupling a proximal portion of a lead to an implantable pulse generator.

FIG. 12C illustrates one embodiment of an improved coupling element for electrically coupling a proximal portion of a lead to an implantable pulse generator.

FIG. 13 illustrates an embodiment of an improved fixation element for an implantable pulse generator and an improved implantable pulse generator for interacting therewith.

FIG. 14 illustrates an implantable pulse generator with an internal battery with a connection interrupt element.

FIG. 15 illustrates an embodiment of a neuromodulation system for an adjacent level revision procedure.

FIG. 16 illustrates an embodiment of a neuromodulation system for a bilateral minimally invasive spinal procedure.

FIG. 17 illustrates an embodiment of the present invention wherein a first neuromodulation system is implanted at a spinal treatment site to deliver a neuromodulation therapy to a first set of one or more nerve targets or anatomical targets.

FIG. 18 illustrates an embodiment of a neuromodulation system in accordance with the present invention wherein the neuromodulation system is implanted at a spinal treatment site in combination with an interspinous device.

### DETAILED DESCRIPTION OF THE INVENTION

Various embodiments of the present invention are described below with reference to the related drawings. The various neuromodulation systems and improvements described below are implantable at a spinal treatment site. Further, the various neuromodulation systems and improvements are implantable at the spinal treatment site in combination with another spinal treatment procedure, such as a spinal fixation procedure, spinal decompression procedure or other spinal implants and procedures performable at a spinal procedure site.

This application incorporates by reference in its entirety the contents of U.S. Provisional Patent Application No. 16/793,319, filed Jan. 16, 2019 and titled NEW NEUROMODULATION THERAPIES AND IMPROVED NEUROMODULATION SYSTEMS.

### Neuromodulation Therapy: Neuromodulation of the Nerves that Innervate the Facet Joint for Treatment of Axial Pain

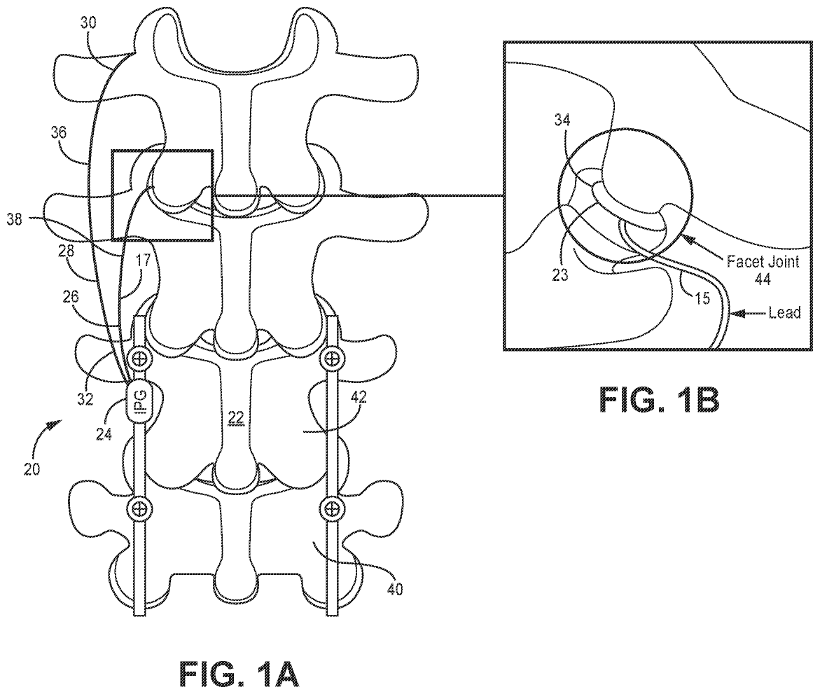

FIGS. 1-3 illustrate embodiments of a neuromodulation system for the treatment of axial pain by innervation of the facet joint.

The facet joints are the connections between the bones of the spine. The nerve roots pass through these joints to go from the spinal cord to the arms, legs and other parts of the body.

These joints also allow the spine to bend and twist, and they keep the back from slipping too far forward or twisting without limits. Like the knee joint, they have cartilage to allow smooth movement where two bones meet. The joints are lined with the synovium and have lubricating joint fluid.

The vertebral bodies are stacked one on top of another to form the entire structure of the spine. On each side of the vertebral bodies are tiny joints called facet joints. Facet Joint Syndrome is a condition in which arthritic change and inflammation occur, and the nerves to the facet joints convey severe and diffuse pain. The most common causes of facet joint pain are degeneration trauma.

The pain does not follow a nerve root pattern. It is actually called “referred pain,” as the brain has trouble localizing these internal structures. Patients often complain of pain in a generalized, poorly defined region of the neck or back. There may be some tenderness overlying the involved joints as well. It is usually caused by trauma (auto accident, whiplash, a bad fall) and a degenerated or herniated disc. These all cause the spine to sublux (move out of joint) and the joint capsule to become irritated. It is usually worsened by sudden movements or prolonged episodes of poor posture, (e.g., kneeling in the garden, bending over to lift, or straining to read a book or look at a computer terminal). Many patients find the worst time is at night, when all the muscles relax and the joints grind together. It can be mistaken for a condition called fibromyalgia or for myofascial syndrome. Often, there is an associated spasm of the muscles in the paraspinal region (on either side of the spine), which can further confuse the diagnosis.

Current treatments for axial pain are physical therapy, medications, facet joint or medial branch blocks, and radio frequency facet rhizotomy. These are temporary solutions and have to be retreated every few months. Also, the procedure itself is painful because these treatments require a therapy capable of penetrating through different layers of tissue, such as with a needle, to get to the target.

As shown in FIGS. 1-3, a neuromodulation system 20 can be implanted at the site of a spinal treatment site 22 and during the course of a spinal procedure, such as a spinal decompression or spinal fixation procedure. The neuromodulation system 20 includes an implantable pulse generator 24 and one or more leads 26, 28 electrically coupled to the implantable pulse generator. The leads 26, 28 include a lead body 15 having a proximal portion 32, a central portion 17 and a distal portion 30. The proximal portion 32 is configured to electrically couple the lead to the implantable pulse generator 24. The distal portion 30 includes one or more electrodes 23 for delivering the neuromodulation therapy to a nerve target or anatomical target in therapeutic proximity to the distal portion 30 of the lead 26, 28 and/or the one or more electrodes 23.

In an open spinal procedure, the placement of the leads 26, 28 may occur under direct physical access, i.e., without epidural tunneling from an access site that is distant from the target site, and the resulting direct vision such that the distal portion 30 of the lead 26, 28, having one or more therapy delivery electrodes 23, is placed onto the target site such that the one or more electrodes 23 are in therapeutic proximity to the target nerve 34 that innervates the facet joint 44. The proximal portion 32 of the lead 26, 28 is electrically coupled to the implantable pulse generator 24 such that the neuromodulation therapy is delivered to the target nerve 34. The nerve target 34 may include any nerve target 34 that enervates the facet joints 44 or any anatomical location that enables the neuromodulation of the target nerves 34 that enervate the facet joints 44, for example the medial branches of the dorsal root.

In a minimally invasive spinal procedure, the placement of the leads 26, 28 may occur under direct vision, depending upon the size and location of the introduction site, with direct placement of the leads 26, 28 on, or in therapeutic proximity to, the target site.

Alternatively, the leads 26, 28 may be place by a lead delivery tool in order to achieve proper lead placement during a minimally invasive spinal procedure.

FIG. 1 illustrates a facet joint pain neuromodulation system 20 having a first lead 26 having a unilateral lead pathway defined by the lead body 15 that extends from an implantable pulse generator 24 positioned at the spinal treatment site 22 to a unilateral target nerve 34 at a first spinal level 40 and a second lead 28 having a unilateral lead pathway defined by the lead body 15 that extends from the implantable pulse generator 24 to a second spinal level 42 such that the distal portion 30 of first lead 26 and second lead 28 are capable of delivering a neuromodulation therapy to a nerve target 34 that innervates the corresponding facet joint 44.

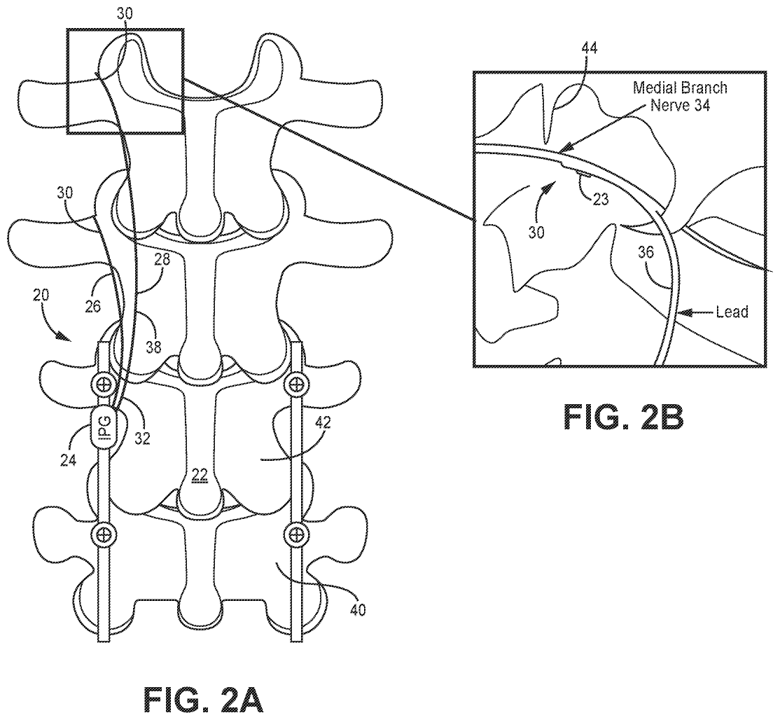

FIG. 2 illustrates a facet joint pain neuromodulation system 20 having medial placement of a first lead 26 and a second lead 28. An implantable pulse generator 24 is implanted within the spinal treatment site 22 and the first and second leads 26, 28 have first and second medial lead pathways defined by the lead body 15 such that the distal portion 30 of each lead 26, 28 innervates a corresponding nerve target 34 that innervates the corresponding facet joint 44.

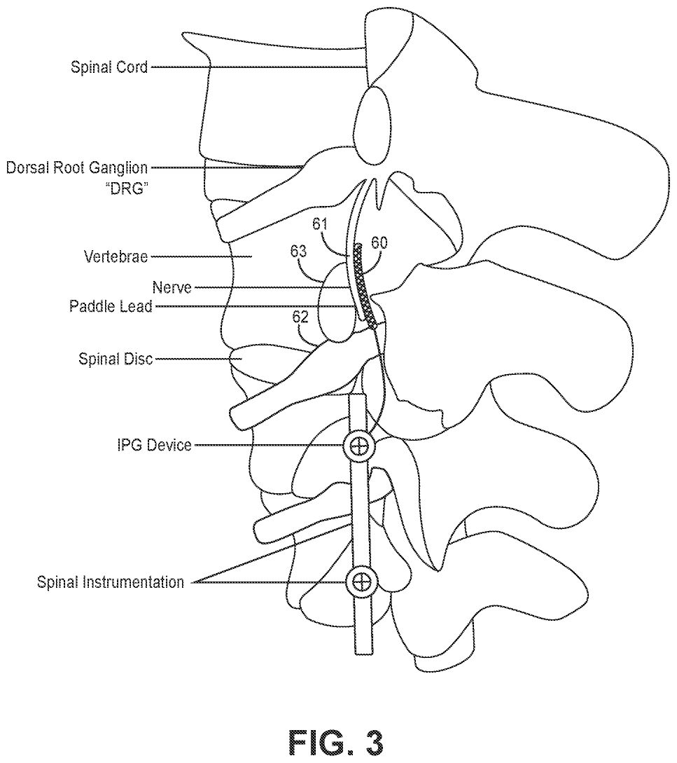

FIG. 3 illustrates a facet joint pain neuromodulation system 20 in accordance with the embodiments described with reference to FIGS. 1-2 wherein the lead is a paddle lead 60 and the lead pathway defined by the lead body 15 is such that the distal portion 30 of the lead 60 is placed directly on the facet joint 44 to provide the neuromodulation therapy to the nerve target 34. In FIG. 3, the paddle lead 60 is positioned on a first portion 61 of the facet joint 44, proximal to the dorsal root 62. Alternatively, the lead may be positioned on the second portion 63 of the facet joint 44 that is distal to the dorsal root 62.

It is understood by those of ordinary skill in the art that the above embodiments are exemplary and that various combinations of lead pathways and lead targets may be employed in the method and system for delivering a facet joint pain neuromodulation therapy. For example, a first lead may have a lateral lead pathway and a second lead may have a medial lead pathway and each lead may have a nerve target at a different spinal level. Furthermore, the neuromodulation therapy may be delivered in the form of electrical stimulation and/or pulsed radio frequency and/or heat and/or cool into the facet joint and/or the nerves innervating them, medial and/or dorsal branches of the dorsal roots.

It is further understood that the neuromodulation method and system for the treatment of axial pain, described above, can be implanted and implemented in combination with other neuromodulation therapies such as those described below. In such case, a first set of one or more leads may be positioned along a lead pathway for delivering a first neuromodulation therapy (such as that described above, or other therapies described below) and a second set of one or more leads may be positioned along a lead pathway for a second neuromodulation therapy (such as dorsal root neuromodulation, or other therapies described either above or below).

### Neuromodulation Therapy: Neuromodulation of the Nerves Inside the Spinal Disc to Treat Discogenic Pain

Discogenic low back pain originates from a damaged intervertebral disc and is a serious medical and social problem, and accounts for 26%-42% of the patients with chronic low back pain. Studies suggested that the degeneration of the painful disc might originate from the injury and subsequent repair of annulus fibrosus.

Chronic low back pain is a serious medical and social problem, and one of the common causes responsible for disability. It is estimated that, in all populations, an individual has an 80% probability of having low back pain at some period during their life time, and about 18% of the population experiences low back pain at any given moment. The expense of treating low back pain is higher than $100 billion each year.

The intervertebral disc is the main joint between two consecutive vertebrae in the vertebral column. Each disc consists of three different structures: an inner gelatinous nucleus pulposus, an outer annulus fibrosus that surrounds the nucleus pulposus, and two cartilage endplates that cover the upper and lower surfaces of vertebral bodies.

Treatment for discogenic low back pain has traditionally been limited to either conservative management or surgical fusion.

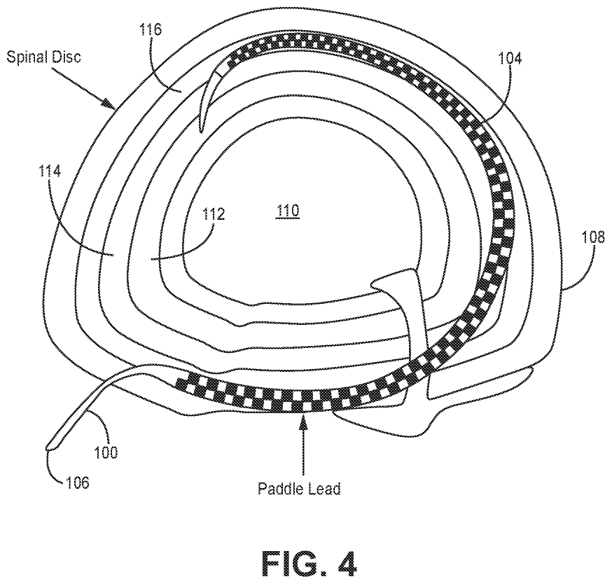

FIG. 4 illustrates an implantable neuromodulation therapy system 120 for the neuromodulation of the target nerves inside the spinal disc 108 to treat discogenic pain.

At a spinal treatment site 22 of an open access spinal procedure, the placement of the leads 100, 102 may occur under direct vision such that the distal portion 30 of the lead 100, 102, having one or more therapy delivery electrodes 23, is placed directly on the target site, e.g, in therapeutic proximity thereto and including but not limited to placed directly on the target site, such that the one or more electrodes 23 are in therapeutic proximity to the target nerve inside the spinal disc 108. The proximal portion 32 of the lead is electrically coupled to the implantable pulse generator 24 such that the neuromodulation therapy is delivered to the target nerve. These nerve targets may include any nerve target or anatomical location capable of providing a neuromodulation therapy to the nerves inside the spinal disc 108.

In a minimally invasive spinal procedure, the placement of the leads may occur under direct vision, depending upon the size and location of the introduction site. Alternatively, the leads may be positioned by an introducer or a lead delivery tool in order to achieve proper lead placement during a minimally invasive spinal procedure.

As shown by way of example in FIG. 4, the distal portion 30 of the lead 100, 102 is placed within the interdiscal space 110 having an inner periphery 112 and an outer periphery 114 where the lead 100, 102 is positionable in either of the inner periphery 112 or outer periphery 114 of the interdiscal space 110. As shown, the lead 100, 102 is a paddle lead having a lead pathway, defined generally by the lead body, that extends along an outer periphery 114 of the interdiscal space 110 to provide the neuromodulation therapy to the nerve target.

It is understood by those of ordinary skill in the art that the above embodiment is exemplary and that combinations of lead pathways and lead targets may be employed in the method and system for delivering an interdiscal neuromodulation therapy. Furthermore, the neuromodulation therapy may be delivered in the form of electrical stimulation and/or pulsed radio frequency and/or heat and/or cool into the facet joint and/or the nerves innervating them, medial and/or dorsal branches of the dorsal roots.

It is further understood that the method and system for the interdiscal neuromodulation, described above, can be implanted and implemented in combination with other neuromodulation therapies such as those described previously, and those described below. In such case, a first set of one or more leads may be positioned along a lead pathway for delivering a first neuromodulation therapy (such as interdiscal neuromodulation, or other therapies described above or below) and a second set of one or more leads may be positioned along a lead pathway for a second neuromodulation therapy (such as dorsal root neuromodulation or other therapies described either above or below).

### Neuromodulation Therapy: Neuromodulation of Muscle Nerves to Minimize Atrophy and Reduce Pain

Muscle atrophy and pain are common problems following spinal surgery. These symptoms may lead to delayed healing and increased use of pain medication, including possibly opiates. Multifidi and rotatores muscles comprise the deepest layer of paraspinal muscles and are often thought to be responsible for fine control of the rotation of vertebrae. They exist throughout the entire length of the spinal column and the multifidi also broadly attach to the sacrum after becoming appreciably thicker in the lumbar region.

Muscle strains and sprains are the most common causes of low back pain. The back is prone to this strain because of its weight-bearing function and involvement in moving, twisting and bending. Lumbar muscle strain is caused when muscle fibers are abnormally stretched or torn. Lumbar sprain is caused when ligaments, the tough bands of tissue that hold bones together, are torn from their attachments. Both of these can result from a sudden injury or from gradual overuse. A doctor may recommend physical therapy. The therapist will perform an in-depth evaluation, which combined with the doctor's diagnosis, will dictate a treatment specifically designed for patients with low back pain. Therapy may include pelvic traction, gentle massage, ice and heat therapy, ultrasound, electrical muscle stimulation and stretching exercises. Pain medication and muscle relaxants may also be beneficial in conjunction with the physical therapy.

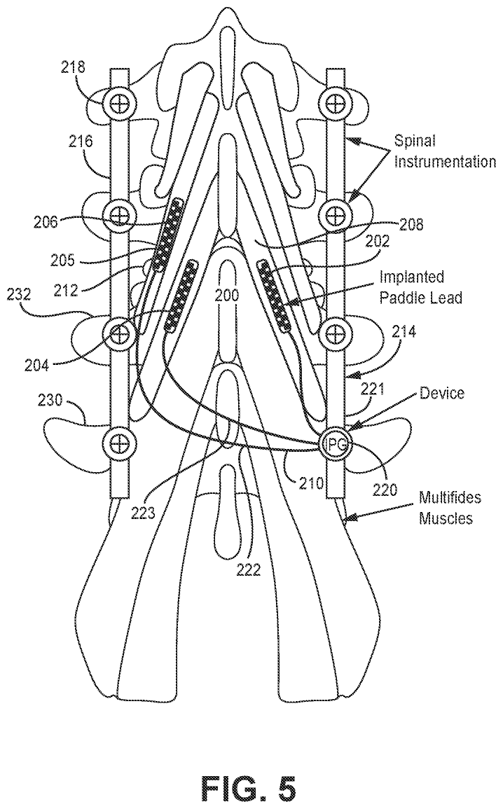

FIG. 5 illustrates an implantable neuromodulation therapy system 201 for the neuromodulation of muscle nerves.

In an open access spinal procedure, the placement of the lead or leads 202, 202, 204 may occur under direct vision at the spinal treatment site 200 such that the distal portion 212 of the lead 202, 204, 205, having one or more therapy delivery electrodes 206, is placed such that the one or more electrodes 206 are placed directly on, or in therapeutic proximity to, the target nerve 208. The proximal portion 210 of the lead 202, 204, 206 is electrically coupled to the implantable pulse generator 220 such that the neuromodulation therapy is delivered to the target nerve 208. These nerve targets may include any nerve target or anatomical location capable of providing a neuromodulation therapy to the target muscle nerves.

In a minimally invasive spinal procedure, the placement of the leads 202,204, 206 may occur under direct vision and with direct placement on, or in therapeutic proximity to, the target site, depending upon the size and location of the introduction site. Alternatively, the leads may be positioned by an introducer or lead delivery tool in order to achieve proper lead positioning during a minimally invasive spinal procedure.

As shown in FIG. 5, a neuromodulation system 201 is implanted at a spinal treatment site 200 that includes an implanted spinal fixation system 214 having a pair of rods 216 and corresponding set of pedical screws 218 fixating the rods 216 to the spinal treatment site 200. An implantable pulse generator 220 is positioned at the spinal treatment site 200 and a set of first, second and third leads 202, 204, 205 extend from the implantable pulse generator 220 such that the corresponding lead body of each lead 20, 204, 206 defines a corresponding first, second and third lead pathway. A distal portion 212 of each lead 202, 204, 205 is positioned in therapeutic proximity to a target nerve 208. Such target nerve 208 may preferably be a multifidi muscle or other spinal muscle groups or para-spinal groups.

In the present embodiment, first and second leads 202, 204 define lead pathways that are medial to lateral such that a distal portion 212 of corresponding first and second leads 202, 204 are positioned on corresponding first and second muscle nerve targets 208 at a first and second spinal levels 230, 232. Third lead 205 defines a lateral lead pathway that defines a lead pathway such that a distal portion 212 of third lead 205 is positioned at a first spinal level 230 such that second lead 204 and third lead 205 provide a bi-lateral muscle nerve target stimulation at the same spinal level 230.

It is understood by those of ordinary skill in the art that the above embodiment is exemplary and that combinations of lead pathways and lead targets may be employed in the method and system for delivering a neuromodulation therapy to a muscle nerve target. Furthermore, the neuromodulation therapy may be delivered in the form of electrical stimulation and/or pulsed radio frequency and/or heat and/or cool into the nerve targets.

It is further understood that the method and system for the neuromodulation, described above, can be implanted and implemented in combination with other neuromodulation therapies such as those described previously, and those described below. In such case, a first set of one or more leads may be positioned along a lead pathway for delivering a first neuromodulation therapy (such as muscle nerve neuromodulation, or other therapies described above or below) and a second set of one or more leads may be positioned along a lead pathway for a second neuromodulation therapy (such as dorsal root neuromodulation or other therapies described either above or below).

### Neuromodulation Therapy: Neuromodulation of Spinal Tracts Targeting Lateral and Anteriolateral Parts of the Spinal Cord

Delivering energy to the deep fibers of the spinal cord has been a great challenge. Current approaches for the use of spinal cord stimulation for treatment of chronic pain include epidural spinal cord stimulators and dorsal root ganglion stimulators, also, surgical and ablative cordotomies (for cancer pain). However, these approaches utilize the posterior epidural space for lead placement and the neuromodulation energy does not go deep enough into the spinal cord. Also, the surgical and ablative cordotomies are irreversible and cause significant side effects such as motor weakness and bladder control problems. It would be advantageous to provide a spinal cord stimulation system and procedure that enables stimulation of the spinal tracts for improved delivery of neuromodulation energy.

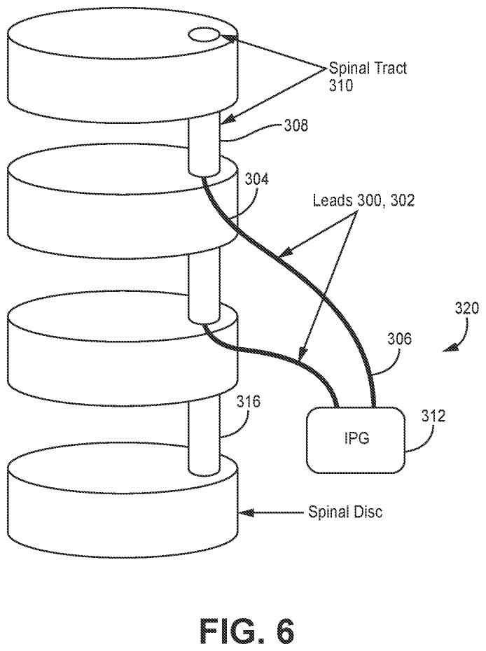

FIG. 6 illustrates an implantable neuromodulation therapy system 320 enabling lead placement adjacent to lateral and anteriolateral parts of the spinal cord in order to modulate spinal tracts.

In an open access spinal procedure, the placement of the lead or leads may occur under direct vision such that the distal portion of the lead 300, 302, having one or more therapy delivery electrodes 308, is placed such that the one or more electrodes 308 are placed directly on, or in therapeutic proximity to, the target nerve of the spinal cord. The proximal portion 306 of the lead 300, 302 is electrically coupled to the implantable pulse generator 312 such that the neuromodulation therapy is delivered to the target nerve 310.

In a minimally invasive spinal procedure, the positioning of the lead or leads may occur under direct vision, depending upon the size and location of the introduction site. Alternatively, the leads may be positioned by an introducer or lead delivery tool in order to achieve proper lead placement during a minimally invasive spinal procedure.

In either direct vision open procedure or minimally invasive implant methods, the lead pathway results in the distal portion 304 of the lead 300, 302 being located adjacent to lateral and anteriolateral parts of the spinal cord, i.e., in therapeutic proximity to the nerve target 310, in order to enable delivery of a neuromodulation therapy to the nerve target 310, preferably the spinal tracts.

For the transforaminal approach, the lead 300, 302 has to pass through neuroforamen in proximity to the dorsal root 314 and into the anteriolateral part of the spinal cord 316. The lead 300, 302 could also be placed intraoperatively, after performing a laminectomy, through a lead pathway defined by a medial or lateral approach providing access that may be resulting from, or improved by, the laminectomy. As the skilled artisan certainly knows, laminectomy is a type of surgery in which a surgeon removes part or all of the vertebral bone (lamina). This helps ease pressure on the spinal cord or the nerve roots that may be caused by injury, herniated disk, narrowing of the canal (spinal stenosis), or tumors. See, e.g., https://www.hopkinsmedicine.org/health/treatment-tests-and-therapies/laminectomy.

This implantable neuromodulation system allows for the delivery of energy to target structures that cannot be achieved with current approaches. For example, the spinothalamic tract. Additionally, a reversible cordotomy is made possible with this system and method while eliminated the side effects of existing approaches.

It is understood by those of ordinary skill in the art that the above embodiment is exemplary and that combinations of lead pathways and lead targets may be employed in the method and system for delivering a neuromodulation therapy to a spinal tract or specific target structures of the spinal tract. Furthermore, the neuromodulation therapy may be delivered in the form of electrical stimulation and/or pulsed radio frequency and/or heating and/or cooling and/or ablative therapies.

It is further understood that the method and system for the neuromodulation, described above, can be implanted and implemented in combination with other neuromodulation therapies such as those described previously, and those described below. In such case, a first set of one or more leads may be positioned along a first set of lead pathways for delivering a first neuromodulation therapy and a second set of one or more leads may be positioned along a second set of lead pathways for delivering a second neuromodulation therapy.

### Neuromodulation Therapy: Neuromodulation of the Interbody Space for Stimulation of Bone Growth

Spinal fixation procedures are performed with the expectation that the interbody space between adjacent spinal levels will lead to spinal fusion resulting from the formation of bone in the interbody space. In an appreciable number of cases, non-fusion may occur. Non-fusion is more likely to occur in patients who are smokers, diabetic or obese or in cases of a multi-level fusion.

Existing solutions include an implantable spinal fusion stimulator from Biomet Spine sold under the product names of the SpF PLUS-Mini and SpF-XL lib. This spinal fusion stimulator includes a pair of mesh cathodes implanted in the lateral gutters of the spine. This spinal fusion stimulator does not directly target the interbody space, where it is desired that the fusion occurs. Instead, one or more intervening anatomical structures may be found in the space between the mesh cathodes and the target interbody space, thereby affecting the effectiveness of the therapy.

Other, non-implantable stimulators exist that are designed to stimulate bone growth for various orthopedic treatments and targets. Non-implantable stimulators face problems of patient compliance with regard to treatment times, device placement and device settings. Such non-implantable stimulators also have to overcome the challenge of intervening anatomical structures in the space between the non-implantable stimulator energy source and the target interbody space.

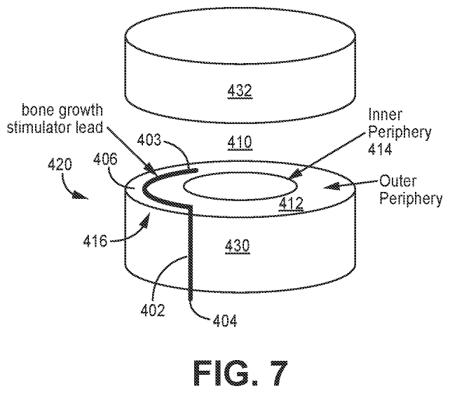

FIG. 7 illustrates a neuromodulation system 420 for targeting the interbody space 410 for the stimulation of bone growth.

In an open access spinal procedure, the placement of the lead or leads 402 may occur under direct vision at the spinal treatment site 400 such that the distal portion 406 of the lead 402, having one or more therapy delivery electrodes 403, is placed such that the one or more electrodes 403 are directly on, or in therapeutic proximity to, the target interbody space 410. The proximal portion 404 of the lead 402 is electrically coupled to the implantable pulse generator 408 to enable delivery of the neuromodulation therapy to the target interbody space 410 via the electrodes 403.

In a minimally invasive spinal procedure, positioning of the lead or leads 402 may occur under direct vision, depending upon the size and location of the introduction site. Alternatively, the lead or leads may be positioned by an introducer or lead delivery tool in order to achieve proper lead placement during a minimally invasive spinal procedure.

The interbody space 410 may have an outer periphery 412 defined by the outer periphery of each of the adjacent vertebral levels 430, 432, and further defines an inner periphery 414 defined as the area proximal to the spinal cord 440 relative to the outer periphery 412. The distal portion 406 of the lead 402 may define a portion of a lead pathway wherein the lead 402 extends through a portion of the outer periphery 412 of the interbody space 410 in a spiral-like pathway.

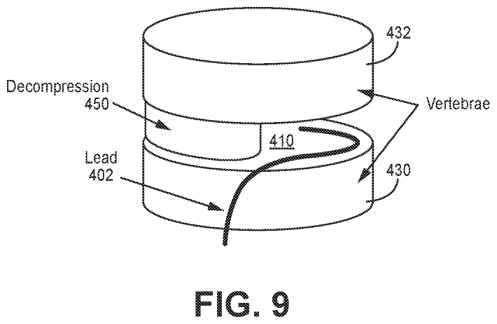

Alternatively, as shown in FIG. 9, one or more leads 402 may be placed to define a lead pathway such that a distal portion 406 of each of the one or more leads 402 has a generally linear pathway wherein the pathway extends on one or the other lateral side of the spinal cord 440, and more preferably in the outer periphery 412 of the interbody space 410.

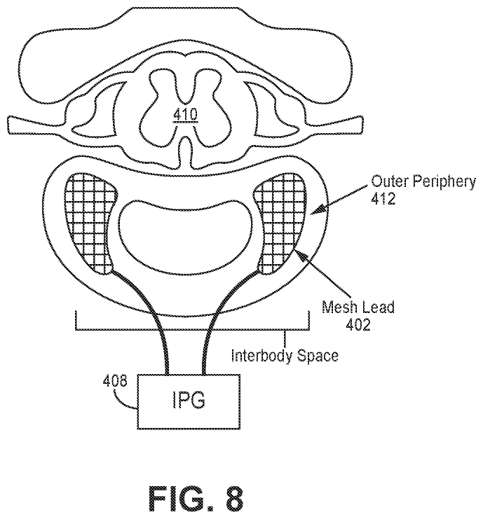

In the neuromodulation systems of FIGS. 7 and 8, the delivery of neurostimulation energy from an implantable pulse generator 408 electrically coupled to the one or more leads 402 would be selectively configured such that only the electrodes 403 that are therapeutically positioned within the interbody space 410 are activated and that any electrodes 403. Other electrodes 403 that are not therapeutically positioned, would be deactivated so as not to deliver an electrical stimulation therapy, such deactivated electrodes 403 would include any electrode that would cause irritation to a non-target nerve or such as an electrode that would cause neuromodulation energy to be delivered to the implanted fixation device 420 such as the rods 422 or pedical screws 424.

As shown in FIG. 9, the implantable neuromodulation system 420 is implanted using a minimally invasive procedure in combination with a spinal decompression procedure. In this embodiment, a decompression element 450 is positioned within a portion of the interbody space 410 and a lead or leads 402 of the neuromodulation system define a lead pathway similar to that described with reference to FIG. 8 or 9 such that the distal portion 406 of the lead 402 is capable of delivering a neuromodulation therapy to an outer periphery 412 of the interbody space 410 for the stimulation of bone growth.

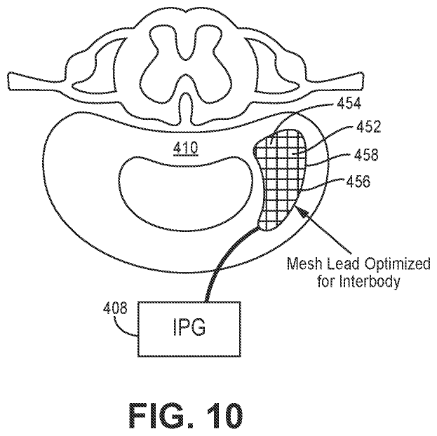

FIG. 10 illustrates a mesh lead 452 design wherein the distal portion 406 of the mesh lead 452 has a mesh shape to provide a broader surface area for delivery of neuromodulation energy to the interbody space 410. The mesh lead 452 has a generally planar contact surface on a first planar surface 454 and second planar surface 456 whereas the side portions 458 of the mesh lead 452 are of a relatively small profile to facilitate placement within the interbody space 410. The mesh lead 452 enables placement and coverage within the interbody space 410 and the delivery of neuromodulation therapy across a greater surface area of the interbody space 410.

The leads of any of the embodiments of FIGS. 7-10 may alternatively be positioned in the lateral gutters of the spine or may be positioned in both the interbody space and the lateral gutters in any combination desirable for the stimulation of bone growth. The lateral gutters may additionally be packed with corticocancellous bone graft on and around the leads placed therein, as may be the interbody space. The leads can be designed and positioned to maximize contact with live bone both on the lateral gutters and/or in the interbody space so as to maximize effectiveness of the neuromodulation therapy.

It is understood by those of ordinary skill in the art that the above embodiments relating to neurostimulation of the interbody space are exemplary and that combinations of lead pathways and lead targets and lead designs may be employed in the method and system for delivering a neuromodulation therapy to an interbody space. Furthermore, the neuromodulation therapy may be delivered in the form of electrical stimulation and/or pulsed radio frequency and/or heating and/or cooling and or ablation therapies into the nerve targets.

It is further understood that the method and system for the above embodiments relating to neurostimulation of the interbody space can be implanted and implemented in combination with other neuromodulation therapies such as those described previously, and those described below. In such case, a first set of one or more leads may be positioned along a lead pathway for delivering a first neuromodulation therapy (such as the interbody neurostimulation therapy described above), and a second set of one or more leads may be positioned along a lead pathway for a second neuromodulation therapy (such as dorsal root neuromodulation or other therapies described either above or below).

### Neuromodulation: Acute Neuromodulation Therapy System

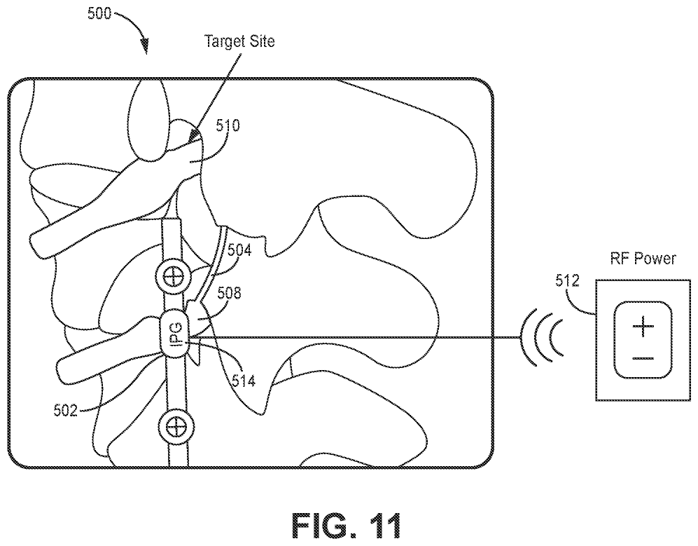

FIG. 11 illustrates an embodiment of an acute neuromodulation system for any of the various neuromodulation therapies described above. The neuromodulation system includes and implantable pulse generator having an energy delivery means and one or more leads having a distal portion and a proximal portion. The proximal portion of the leads are electrically couple-able to the implantable pulse generator and the distal portion of the leads are positionable at, near or in therapeutic proximity to a target nerve or anatomical target.

The implantable pulse generator does not have a battery or other internal power source but instead is powered by an external power source via radiofrequency (RF) coupled induction. This external power source allows for selective delivery of a neuromodulation therapy when the external power source is inductively coupled to the implantable pulse generator. The neuromodulation therapy may include a continuous delivery of power over a predetermined time period or a prescription of time intervals over a period of time during which to power the implantable pulse generator and deliver the neuromodulation therapy.

An acute neuromodulation therapy in accordance with the embodiment shown in FIG. 11 may include any of the therapies described above, for example the bone growth stimulation therapy as described with reference to FIGS. 7-10 above but instead of using an implantable pulse generator with an internal power source, using the implantable pulse generator and external power source described with reference to FIG. 11. The therapy may be delivered periodically, one or more times per day for a specified period of time, and repeated one or more days, weeks, or months for a specified number of days, weeks or months. Such therapy delivery schedule may, of course, include intermittent or periodic changes in the therapy delivery timing and attributes.

The implantable pulse generator of FIG. 11 may further include absorbable electronics 514 so as to eliminate any requirement for explant of the implantable pulse generator and/or to avoid any complications that may arise as the result of an inactive implantable pulse generator 502 remaining implanted in a patient anatomy.

### Improved Lead—IPG Coupling Elements

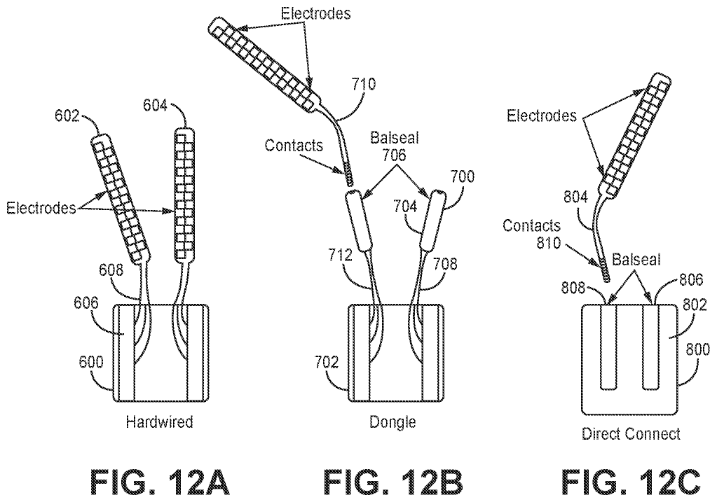

FIGS. 12A-12C illustrate various embodiments of improved coupling elements for electrically coupling a proximal portion of a lead to an implantable pulse generator. The improved coupling elements may be incorporated in any of the above neuromodulation therapies as should be reasonably understood to those of ordinary skill in the art.

FIG. 12A illustrates an implantable pulse generator 600 in combination with leads 602, 604 having a hardwired connection 606 therebetween. The proximal portion 608 of the leads are fixedly secured to the implantable pulse generator via the hardwired connection 606, thus requiring the implantation of the leads 602, 604 and implantable pulse generator 600 simultaneously. This embodiment enables a smaller volume of space to be taken up by the implantable pulse generator 600 relative to existing commercial implantable pulse generators.

FIG. 12B illustrates one or more dongle connectors 700 extending from the implantable pulse generator 702. In the present embodiment, the dongle connector 700 has a distal portion 702 that is hard-wired into the implantable pulse generator and a distal portion 704 that includes a seal 706 for receiving a proximal portion 708 of a lead 710 therein and thereby electrically coupling the implantable pulse generator 702 to the lead 710 via the dongle connector 700. The dongle connector 700 may incorporate a bendable central portion 712 and/or comprise a shape memory material or capability such that the dongle connector 700, or a portion of the dongle connector 700, acts as an anchor and/or as a strain relief function when coupled to a lead 710. The dongle connector 700 acts to anchor and maintain the position of the lead 710 so as to ensure ongoing delivery of the neuromodulation therapy by the lead 710 to the target nerve or anatomy. The seal 706 created between the dongle connector 700 and the proximal portion 708 of the lead 710 received by the dongle connector 700 may be a Bal Seal™ or other such spring energized seals.

FIG. 12C illustrates an implantable pulse generator 800 with a direct connect housing 802 for receiving one or more leads. The implantable pulse generator 800 has one or more receptacles 806 with electrically couplable electrodes 808 for receiving a proximal portion 810 of a lead 804 with corresponding electrically couplable electrodes 808 so as to create an electrical coupling between the implantable pulse generator 800 and lead 804 when the proximal portion 810 of the lead 804 is received within the receptacle 806.

### Improved IPG Fixation Elements

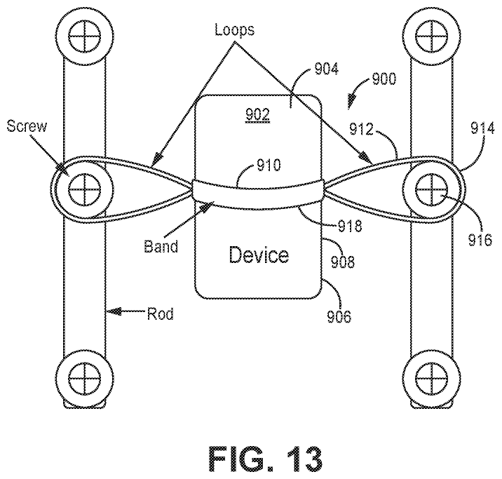

FIG. 13 illustrates an embodiment of an improved fixation element 900 for an implantable pulse generator 902.

The implantable pulse generator 902 has body comprising first and second major side panels 904, 906 and side sections 908 extending therebetween.

The fixation element 900 is a flexible and elastically extendible band 910 that is capable of engaging at least a portion of the implantable pulse generator 902 via a compression force. The fixation element 900 may further include one or more additional fixation loops 912 for elastically extending to a target anchor site 914, such as a bone in a patient anatomy or a pedical screw in a spinal implant. The fixation loops 912 anchor the implantable pulse generator 902 to the target anchor site 914 in combination with the fixation loop 912. As shown, the fixation element 900 includes a first and second fixation loop 912, each anchored to a corresponding first and second pedical screw 916. Any number of fixation loops 912 may be incorporated in the fixation element 900 and any number and combination of target anchor sites 914 may be elastically engaged by the fixation loop 912 in accordance with this embodiment.

The implantable pulse generator 902, as shown, further includes a recessed contour 918 defined within at least one of the major side panels 904, 906 and is configured to receive at least a portion of the band 910 of fixation element therein 900. An implantable pulse generator 902 may have any number of such recessed contours 918 defined across any surface 904, 906, 908 of the implantable pulse generator 902 in accordance with this embodiment.

### IPG with Long-Term Off Mode

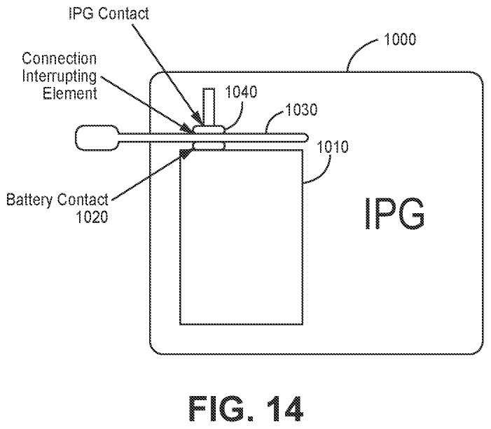

FIG. 14 illustrates an implantable pulse generator 1000 with an internal battery 1010. The internal battery 1010 has one or more electrical coupling connections 1020 and at least one of the coupling connections 1020 has a connection interrupt element 1030 that is repositionable between a first position and a second position. In the first position, the connection interrupt element is placed between the coupling connection 1020 and the corresponding contact 1040 of the implantable pulse generator 1000 such that the implantable pulse generator 1000 is set to a “long-term-off” mode without dissipating the energy stored in the battery and rendering the battery unusable. When the implantable pulse generator 1000 is used again at a later date, the connection interrupt element 1030 is repositionable to a second position. In the second position, the connection interrupt element 1030 no longer interferes with the connection between the battery coupling connection 1020 and corresponding contact 1040 of the implantable pulse generator 1000, allowing electrical coupling to occur therebetween. As a result, the battery is able to be recharged by external inductive charging even after being in a long-term off mode and the implantable pulse generator 1000 is able to draw energy from the battery 101 via the coupling connection 1020.

### Improved Minimally Invasive Implant Method

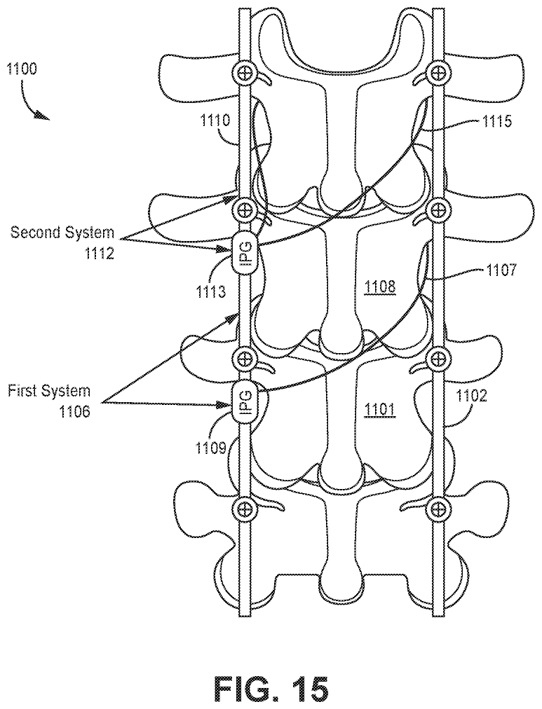

FIG. 15 illustrates an embodiment of a neuromodulation system 1100 for an adjacent level revision procedure. A first spinal procedure is performed at a first spinal level 1101. The first spinal procedure is a first spinal implant 1102 such as spinal fixation procedure or a spinal decompression procedure. The first spinal procedure further includes first neuromodulation system 1106 being implanted during the first spinal procedure. First neuromodulation system 1106 includes an implantable pulse generator 1109 and one or more leads 1107.

At a later date, a second spinal procedure is performed at an adjacent spinal level 1108, such as a revision procedure. During the second spinal procedure a second spinal implant 1110 is placed at the adjacent spinal level 1108, such as a second spinal fixation implant. Also during the second spinal procedure, a second neuromodulation system 1112 is implanted at or near the adjacent spinal level 1108. Second neuromodulation system includes an implantable pulse generator 1113 and one or more leads 1115.

First and second neuromodulation systems 1106, 1112 each may provide a neuromodulation therapy or combination of therapies in accordance with the various embodiments described herein.

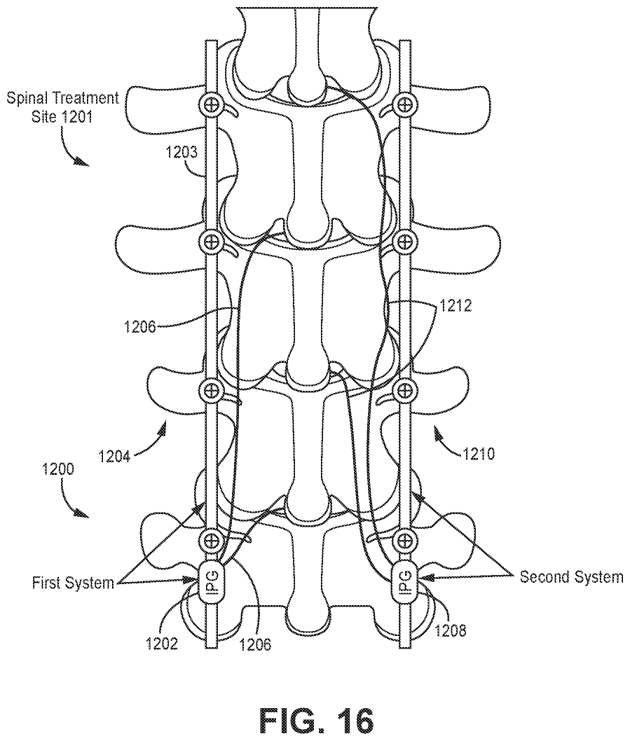

FIG. 16 illustrates an embodiment of a neuromodulation system 1200 for a bilateral minimally invasive spinal procedure. A spinal treatment site 1201 is shown, having a spinal fixation device 1203 implanted, preferably by a minimally invasive implant method. The neuromodulation system includes a first implantable pulse generator 1202 implanted by a minimally invasive procedure on a first lateral side 1204 of the spinal procedures site and a first set of one or more leads 1206 electrically coupled to the first implantable pulse generator 1202. The neuromodulation system 1200 further includes a second implantable pulse generator 1208 implanted by a minimally invasive procedure on a second lateral side 1210 of the spinal procedures site and includes a second set of one or more leads 1212 electrically coupled to the second implantable pulse generator 1208.

The leads 1206, 1212 may follow any number of desired lead pathways including but not limited to extending from the corresponding implantable pulse generator 1202, 1208 to a nerve target or anatomical targets on the same lateral side as the corresponding pulse generator 1202, 1208 or extending to any medially located target or extending to a target on the opposing lateral side.

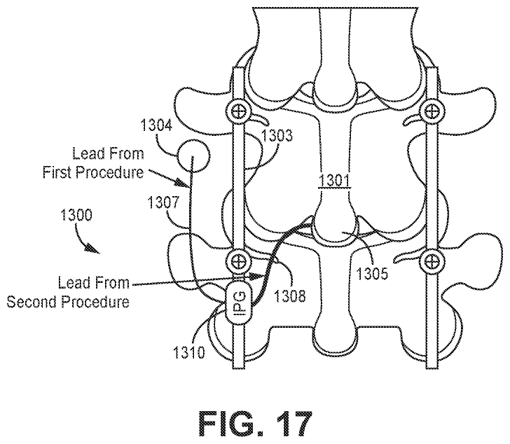

FIG. 17 illustrates an embodiment of the present invention wherein a neuromodulation system 1300 is implanted at a spinal treatment site 1301 to deliver a neuromodulation therapy to a first set of one or more nerve targets or anatomical targets 1304 and a second set of one or more nerve targets 1305 of a different type of nerve or anatomical target than the first set 1304. A spinal treatment site 1301 is shown, having a spinal fixation device 1303 implanted at the spinal treatment site 1301, preferably by a minimally invasive implant method.

The first set of one or more leads 1307 may be placed at the spinal treatment site 1301 during the same spinal procedure in which the spinal fixation device 1303 is implanted. A distal portion of the first set of leads 1307 are placed in therapeutic proximity to the first set of one or more targets 1304 and a proximal portion of the leads 1307 are electrically coupled to the implantable pulse generator 1310 so as to enable delivery of a neuromodulation therapy to the first set of targets 1304.

The second set of one or more leads 1308 may be placed at the spinal treatment site 1301 during the same spinal procedure as well. Alternatively, the second set of one or more leads 1308 may be placed at a time after the spinal procedure is completed. The second set of nerve targets 1305 can be accessed by a minimally invasive procedure or open back procedure wherein a distal portion of each of the second set of one or more leads 1308 corresponding to the one or more second set of targets 1305. Each of the leads 1308 are placed such that a distal portion of the lead 1308 is placed in therapeutic proximity to the second set of one or more targets 1305 and the proximal portion of the lead is electrically coupled to the implantable pulse generator so as to allow the delivery of a neuromodulation therapy to the second set of nerve targets 1305.

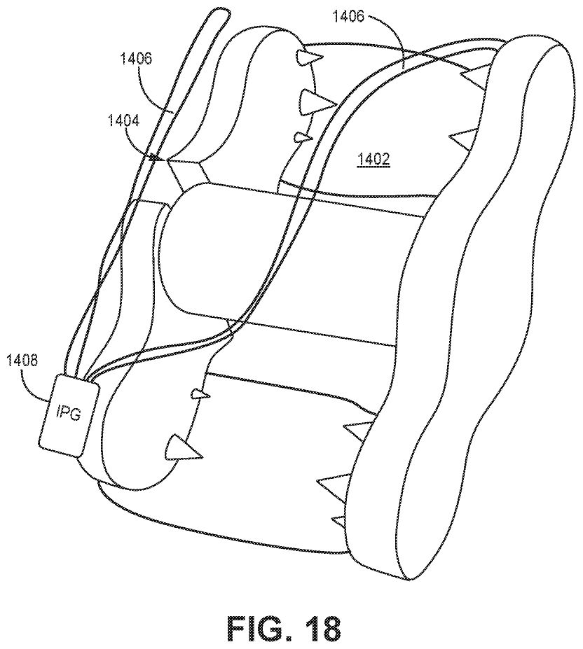

FIG. 18 illustrates an embodiment of a neuromodulation system 1400 in accordance with the present invention wherein the neuromodulation system 1400 is implanted at a spinal treatment site 1402 in combination with an interspinous device 1404 such as the Polyaxial ZIP® ISP by Aurora Spine. The interspinous device 1404 is implanted at a first spinal level at a spinal treatment site and the neuromodulation system is implanted during the same spinal procedure at the same or an adjacent or nearby spinal level. As with this embodiment and all aforementioned embodiments, the neuromodulation system 1400 is implanted according to patient needs and/or physician preferences such that the one or more leads 1406 follow a lead pathway such that a distal portion of the one or more leads 1406 are in therapeutic proximity to a target nerve or anatomical site and the proximal portion of the leads are electrically coupled to the pulse generator 1408.

As with this embodiment and all other aforementioned embodiments, the lead may be connected to the implantable pulse generator by the various means of connecting described with reference to FIGS. 12A-12C and the implantable pulse generator may be anchored to the interspinous device via the fixation elements described above with reference to FIG. 13 and may include, as desired, any other elements described in the embodiments above.

The description of the invention and is as set forth herein is illustrative and is not intended to limit the scope of the invention. Features of various embodiments may be combined with other embodiments within the contemplation of this invention. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A method for treating pain generated by a patient's spinal column with neuromodulation stimulation and spinal decompression procedures, comprising:
identifying one or more targeted spinal level for combined neuromodulation stimulation and two targeted vertebral levels, each level comprising a vertebrae, for spinal decompression;
creating an open access into the resulting spinal treatment site comprising at least one targeted dorsal root ganglion within the identified one or more targeted spinal levels;
placing a neuromodulation system within the spinal treatment site, the neuromodulation system comprising:
an implantable pulse generator,
an electrical lead in operative electrical communication with the implantable pulse generator, wherein the electrical lead is routed from the implantable pulse generator to one of the least one targeted dorsal root ganglion, and
an electrode disposed at a distal end of the electrical lead; and
placing the electrode in therapeutic proximity with the least one targeted dorsal root ganglion;
executing the spinal decompression procedure before or after the placing of the neuromodulation system;
closing the open access; and
delivering a neuromodulation stimulation therapy with the neuromodulation system after before and/or after closing the open access.

2. The method of claim 1, wherein the open access into the spinal treatment site provides direct visual access to the at least one targeted dorsal root ganglion.

3. The method of claim 1, wherein the open access, the placing of the neuromodulation system and the executing of the spinal decompression procedure are all minimally invasive.

4. The method of claim 1, wherein the spinal decompression procedure comprises implanting a decompression element between the vertebrae in the two targeted vertebral levels.

5. The method of claim 4, further comprising providing an interbody space between the vertebrae in the two targeted vertebral levels as a result of the implanting of the decompression element.

6. The method of claim 5, further comprising providing a lead pathway along which the electrical lead is routed, wherein a portion of the lead pathway is disposed within the provided interbody space.

7. The method of claim 1, wherein the neuromodulation system comprises:
a plurality of electrical leads in operative electrical communication with the implantable pulse generator, wherein each electrical lead in the plurality of electrical leads is routed from the implantable pulse generator to the one or more targeted spinal levels; and
an electrode disposed at the distal end of each one of the plurality of electrical leads.

8. The method of claim 7, wherein the open access into the spinal treatment site provides direct visual access to the at least one targeted dorsal root ganglion, and placing each of the electrodes in therapeutic proximity with separate ones of the at least one targeted dorsal root ganglia using the direct visual access provided by the open access.

9. The method of claim 8, wherein the open access into the spinal treatment site further provides direct physical access to each of the at least one targeted dorsal root gangion; and
placing each electrode in therapeutic proximity with separate ones of the at least one targeted dorsal root ganglion using the created direct visual access to the at least one targeted dorsal root ganglion and the created direct physical access to the at least one targeted dorsal root ganglion.

10. The method of claim 8, further comprising delivering a neuromodulation system therapy with the neuromodulation system before closing the open access.

11. The method of claim 8, further comprising delivering a neuromodulation system therapy with the neuromodulation system after closing the open access.

12. The method of claim 7, further comprising routing at least one of the plurality of electrical leads to cross the midline of the patient's spinal column.

13. The method of claim 1, further comprising identifying the midline of the patient's spinal column, and placing the implantable pulse generator along the midline of the patient's spinal column.

14. The method of claim 7, further comprising routing at least one of the plurality of electrical leads without crossing the midline of the patient's spinal column.

15. The method of claim 1, further comprising identifying the midline of the patient's spinal column, and placing the implantable pulse generator along the midline of the patient's spinal column.

16. The method of claim 8, further comprising placing the implantable pulse generator below the placed electrodes.

17. The method of claim 1, further comprising placing the implantable pulse generator on one side of the midline of the spinal column.

18. The method of claim 1, further comprising routing at least one electrical lead to cross the midline of the patient's spinal column.

19. The method of claim 1, wherein the electrode comprises a paddle.

20. The method of claim 1, wherein the implantable pulse generator comprises an internal battery power source.

21. A method for placing a neuromodulation electrical lead in combination with a spinal decompression procedure, comprising:
identifying one or more targeted spinal and two targeted vertebral levels, each level comprising a vertebrae, for combined spinal decompression and placing of the electrical lead;
creating a minimally invasive open access into the resulting spinal treatment site and at least one targeted dorsal root ganglion within the identified one or more targeted spinal levels and ;
placing an electrical lead in operative electrical communication with the implantable pulse generator, wherein the electrical lead is routed to one of the least one targeted dorsal root ganglion, wherein the electrical lead comprises an electrode disposed at a distal end of the electrical lead; and
placing the electrode in therapeutic proximity with the least one targeted dorsal root ganglion;
executing the spinal decompression procedure by implanting a decompression element between the two vertebrae within the two targeted vertebral levels before or after the placing of the neuromodulation system;
closing the open access.

22. The method of claim 21, comprising executing the spinal decompression procedure before placing the electrical lead, wherein the spinal decompression procedure provides an interbody space between the two vertebrae within the two targeted vertebral levels; and
routing the electrical lead through the provided interbody space.

23. The method of claim 21, wherein the minimally invasive open access into the resulting spinal treatment site provides direct visual access to the at least one targeted dorsal root ganglion.

24. The method of claim 23, further comprising using the direct visual access to the at least one targeted dorsal root ganglion to place the electrode in therapeutic proximity with the at least one targeted dorsal root ganglion.
